(19) United States
(12) Patent Application Publication
(10) Pub. No.: US 2024/0188830 A1
(43) Pub. Date: Jun. 13, 2024

(54) RESPIRE 1

(71) Applicant: LeTourneau University, Longview, TX (US)

(72) Inventors: Maxx Frick, Longview, TX (US); Charles Cook, Longview, TX (US); Devon Cuperus, Longview, TX (US); Drake Dunn, Portage, MI (US); Lindsey Greene, Evans, GA (US); Gabriel Hurlbert, Manassas, VA (US); Joon W. Kim, Longview, TX (US); Christin Kelly, Longview, TX (US); Andrew Lang, Palmyra, NE (US); Katherine Le, Longview, TX (US); Sungyub Lew, Chapel Hill, NC (US); Grace Nakano, Celina, TX (US); Sean Nutt, Dallas, TX (US); Alistair Park, Longview, TX (US); Zachary Poorman, West Haven, UT (US); Savannah Orton, Longview, TX (US); Julian Lee, Albuquerque, NM (US); Erin O'Brien, S. Hamilton, MA (US); Ashley DeCock, Natalia, TX (US); Morgan Nix, Houston, TX (US); Evan Kowal, Warsaw, MO (US); Samson Segovia, Forney, TX (US); Ian Crochet, New Iberia, LA (US); Oladimeji Apaokagi, Dallas, TX (US)

(73) Assignee: LeTourneau University, Longview, TX (US)

(21) Appl. No.: 18/135,151

(22) Filed: Apr. 15, 2023

Related U.S. Application Data
(60) Provisional application No. 63/322,298, filed on Mar. 22, 2022.

(51) Int. Cl. A61B 5/00; A61B 5/01; A61B 5/08; A61B 90/50; G06V 40/16

(52) U.S. Cl. CPC A61B 5/0077; A61B 5/0002; A61B 5/015; A61B 5/0816; A61B 5/746; A61B 5/748; A61B 90/50; G06V 40/171; A61B 2560/0431

(57) ABSTRACT

The present invention relates to the use of imaging for non-invasive and non-contact measurement of respiration rates and body temperature. The invention uses a combination of visible imaging, thermal imaging, and machine learning to monitor respiration rate and body temperature. One of the embodiments of the invention allows the invention to be mounted on an articulating arm for use in hospital settings. Another embodiment of the invention allows the invention to be easily portable for use in mobile applications. Further embodiments allow the system to be used remotely making it very applicable for use in isolation or for remote locations.

### CROSS-REFERENCE TO RELATED APPLICATION

The present application claims priority to, and the benefit of, U.S. Provisional Application No. 63/332,298, which was filed on Apr. 19, 2022, and is incorporated herein by reference in its entirety.

### FIELD OF THE INVENTION

The present invention relates generally to the field of medical measurements. More specifically, the present invention relates to the use of imaging for non-invasive measurement of respiration rates and temperature. The invention uses a combination of visible imaging, thermal imaging, and machine learning to monitor respiration rate and body temperature.

### BACKGROUND OF THE INVENTION

Respiration rate is a vital sign in most health assessment situations; unfortunately, current methods produce medical waste and are limited in their use. Current devices require skin contact to measure respiration rate. The requirement of skin contact in the measurement of respiration rate creates medical waste and in some critical trauma situations, wherein the amount of intact skin may be limited, the requirement for skin contact makes the method unusuable. In situations involving possibly contagions, the requirement for skin contact to measure respiration rate is also problematic. Therefore, there is a long-felt need in the art of respiration rate measurements for an accurate and non-invasive rate measurement.

The present invention, Respire I, requires no physical contact, produces no medical waste, and provides accurate, fast, and reliable respiratory data using a combination of thermal and visible light imaging. Visible light imaging combined with machine learning is used to locate a nasal region while thermal imaging of the nasal region is then used to measure respiratory rate and monitor temperature.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later

The invention, Respire I, in one embodiment thereof is a medical monitoring device able to remotely, non-invasively, and without physical contact measure and monitor respiratory rate and body temperature; and is able to alert medical staff to abnormal breathing patterns or changes in body temperature. The invention is comprised of a visible imaging camera, a thermal imaging camera, a processing unit, an input/output device, and software or firmware that incorporates machine learning. The software uses machine learning to effectively identify the nose and mouth area in the visible image. The visible image and the thermal image are then overlaid so as to identify thermal regions related to respiration. The software is configured to measure the thermal changes and calculate a respiration rate. In like manner, to those skilled in the art, the invention can also be applied to other medical measurements such as temperature.

Another embodiment of the invention, a medical monitoring system is disclosed. The system integrates the medical monitoring device into a mobile articulating arm that allows the device to be easily transported and positioned for use in hospital and medical clinic settings. The device has a simple user interface and is mobile and compact allowing it to be used with minimal training in many environments.

In yet another embodiment of the invention, the device is a portable, singular unit powered by a wall outlet. The portable, singular unit can be attached to a common tripod mount or implemented into hospital, clinical, and ambulatory needs utilizing a threaded insert on the base of the unit.

In any and all embodiments of the present invention, the invention can be controlled remotely, allowing for a non-invasive remote process of measuring respiration and body temperature.

### BRIEF DESCRIPTION OF THE DRAWINGS

The embodiments herein will be better understood from the following detailed description with reference to the drawings in which:

FIG. 1 illustrates a schematic representation of the medical monitoring device in accordance with the disclosed architecture;

FIG. 2 is a perspective view of a housing case for the visible imaging camera and the thermal imaging camera;

FIG. 3 is a perspective view of the medical monitoring system in accordance with the disclosed architecture of the invention;

FIG. 4 illustrates a flow diagram of the process for measuring respiration rate in accordance with the disclosed architecture;

FIG. 5 illustrates a further details of the process for measuring respiration rate and body temperature in accordance with the disclosed architecture;

FIG. 6 illustrates a schematic representation of the communication among the components of the medical monitoring device in accordance with the disclosed architecture;

FIG. 7 is an example of the thermal imaging acquired from the device's thermal camera;

FIG. 8 is a view of the full assembly, with the medical monitoring system enclosed in a portable, mobile unit;

FIG. 9 is the full assembly exploded view of the mobile unit;

FIG. 10 is a front and back view of mobile unit;

FIG. 11 provides views of the back panel of the mobile unit;

FIG. 12 is a section view of the front of the mobile unit; and,

FIG. 13 is a section view of the back of the mobile unit.

### DETAILED DESCRIPTION OF THE INVENTION

The innovation is now described with reference to the drawings, wherein like reference numerals are used to refer to like elements throughout. In the following description, for purposes of explanation, numerous specific details are set forth to provide a thorough understanding thereof. It may be evident, however, that innovation can be practiced without these specific details. In other instances, well-known structures and devices are shown in block diagram form to facilitate a description thereof. Several embodiments and their modifications are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, any of the modifications or features described herein with respect to one embodiment may also be applied to another embodiment.

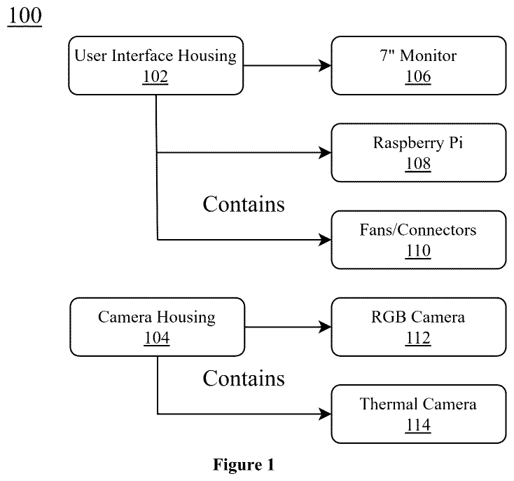

Referring to the drawings, FIG. 1 illustrates a schematic perspective of one embodiment of the invention. The medical monitoring device 100 is comprised of a user interface housing 102 and a camera housing 104. The camera housing 104 is further comprised of a RGB camera 112 whereby a visible image is obtained; and a thermal camera 114, whereby a thermal image is obtained. The user interface housing 102 is further comprised of a processing unit 108 configured to receive the visible image and the thermal image, and a monitor 106 to control the device and display results. The processing unit 108 includes software or firmware configured to identify and track the mouth and nasal region in the visible image. The software or firmware utilizes machine learning to enable the identification of the mouth and nasal region. The coordinates of the mouth and nasal region in the visible image are used to identify the coordinates of the mouth and nasal regions in the thermal image. The software extracts the thermal information from the identified mouth and nasal regions. From the extracted thermal information, the software can monitor thermal changes as a function of time allowing the processing unit to calculate respiration rates, identify abnormal breathing patterns and monitor temperature.

The software on the processing unit 108 includes machine learning components configured to allow the RGB camera 112 to locate and track facial features such as mouth and nasal regions. The coordinates obtained in the visible image are transposed to coordinates in the images obtained by the thermal camera 114. The software could be integrated into the processing unit 108 as firmware.

In variations of this embodiment, the medical monitoring device 100 can be attached to a wall, bed, or any other stationary or mobile item. The RGB camera 112 can be any camera that can capture a visible video image. The thermal camera 114 can be any camera that can capture a thermal video image. The processing unit 108 can be any logic processing unit such as microprocessors, digital signal processors (DSPs), application-specific integrated circuits (ASICs), field programmable gate arrays (FPGAs), multiprocessors such as a raspberry pi, or similar functioning device. The monitor 106 may be any device that allows a user to control the device and display the results. The monitor device 108 may be a keyboard and a display screen, the monitor device may be combined into one touchscreen display or non-touchscreen display. The device may also be operated remotely wherein a computer at a remote location can be used to control the device and monitor outputs.

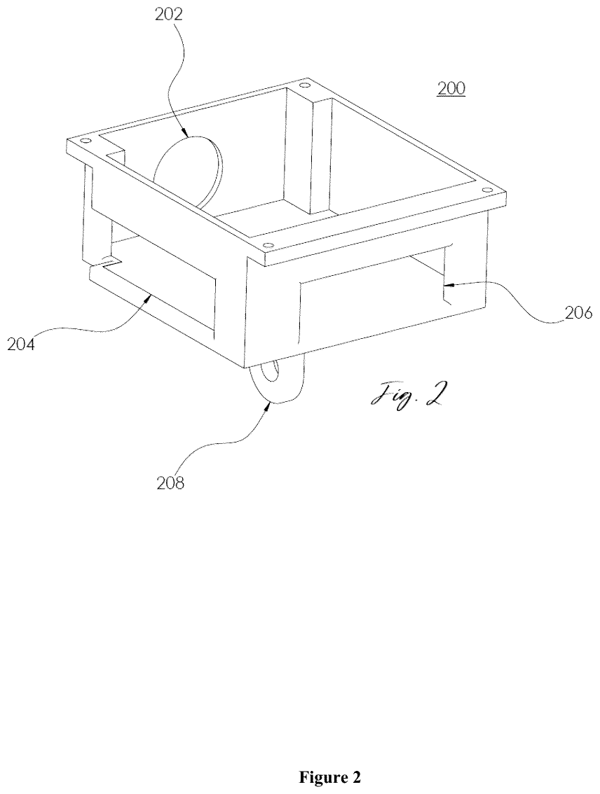

FIG. 2 is a perspective view of an embodiment of the medical monitoring device in which the device 100 of FIG. 1 includes a more sophisticated housing 200 in which the visible camera 112 and the thermal camera 114 are contained. The housing 200 is configured with a first hole 202 and a second hole 204 in through which the visible imaging camera 102 and the thermal imaging camera 104 can obtain images outside of the housing 200. The distance between the first hole 202 and the second hole 204 is used to correlate the coordinates of the visible images obtained by the visible imaging camera 112 and the thermal image obtained by the thermal imaging camera 114; thereby, allowing the processing unit 108 to extract thermal information from thermal image that corresponds to coordinates identified as the mouth and nasal region in the visible image.

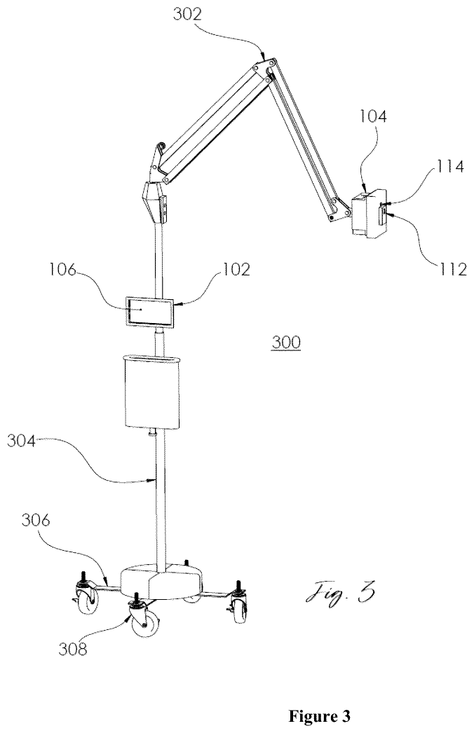

FIG. 3 illustrates a perspective view of another embodiment of the present invention. The medical monitoring system 300 is comprised of all the elements of the medical monitoring device 100. In addition, the medical monitoring system 300 includes an articulating arm 302, a pole 304, a base 306, and at least three casters 308. In the medical monitoring system 300, the elements of the aforementioned medical monitoring device 100 are attached to the articulating arm 302, the pole 304, or the base 306. The medical monitoring system 300 with the articulating arm 302 allows greater flexibility and portability and is specifically configured to meet the needs of hospital and clinical settings. The articulating arm 302 is attached to the pole 304 which is supported by the base 306. The at least three casters 308 allow the medical monitoring system 300 to translate across level surfaces. The medical monitoring system 300 is configured specifically for use in hospitals and clinics. The monitor 106 is attached to the pole 304. In this embodiment the user interface housing 102 of FIG. 1 also contains the cameras 112 and 114, as well as the enclosed Raspberry Pi 108 and is attached to the articulating arm 302.

As a variation of this embodiment, the medical monitoring system 300 may also include the housing 200 containing the visible imaging camera 112 and the thermal imaging camera 114, as shown in FIG. 2. The housing 200 containing visible imaging camera 112 and thermal imaging camera 114 may be attached to the articulating arm 302.

Alternatively, the visible imaging camera 112 and the thermal imaging camera 114 may be independently attached to the articulating arm 302.

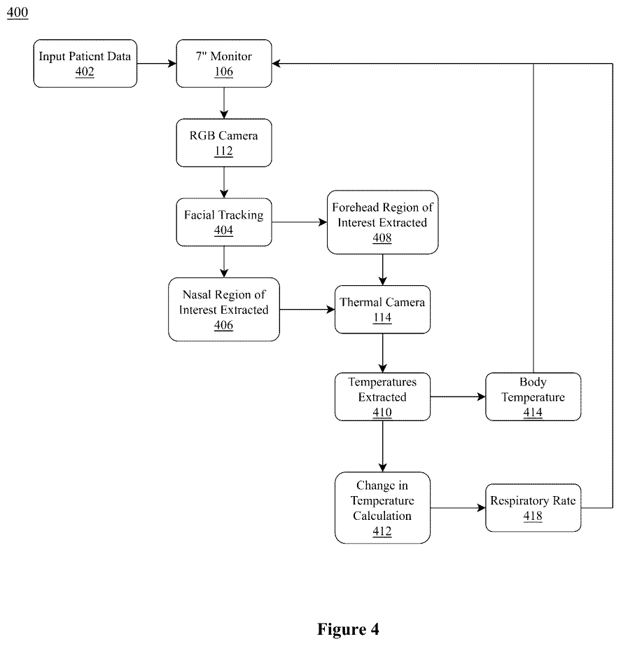

FIG. 4 illustrates yet another embodiment of the present invention, a non-invasive method of monitoring vital signs such as respiration rates and temperature. The method includes inputting data 402 using the monitor 106, shown in FIG. 3, as an input device. Data from the input/output device directs the visible imaging camera 112 and thermal images camera 114 to track and image nasal and forehead regions 404 and allows the extraction of thermal data 410 from the thermal imaging using coordinates from the visible imaging. From the thermal data temperature information can be extracted in step 410 and be correlated to body temperature in step 414. The change in temperature can also be used to calculate respiratory rate 418. All the data and calculation can be displayed on monitor 106 wherein monitor 106 is used as an output device.

An algorithm is implemented to calculate the respiratory rate. The temperature changes are temporarily stored and analyzed by the algorithm. The temperature signal is compared to a threshold of the average temperatures of the signal over a fixed number of data points before a new threshold is calculated. A breath is counted if the temperature signal crosses the threshold bounds in a cyclic fashion.

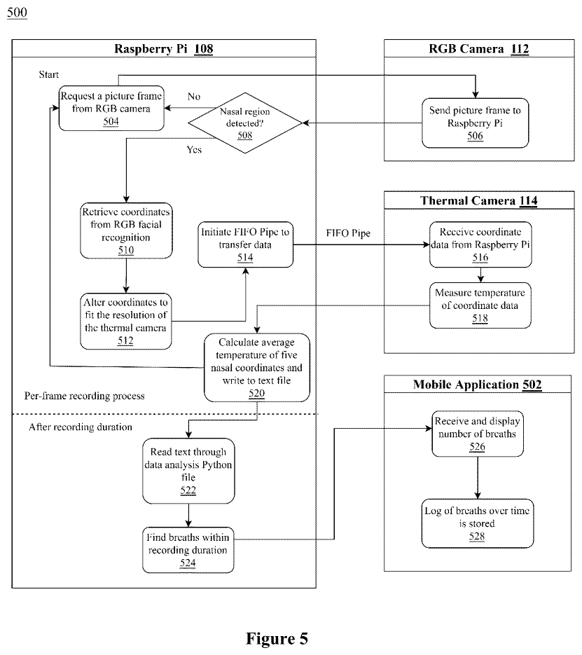

FIG. 5 illustrates further details of the method incorporated into the software. The method includes beginning the recording duration by first starting the software by the processing unit 108, shown in FIG. 1, and requesting, step 504, a picture frame from the RGB camera. In step 508, the image is sent to the processing unit 108 to detect the nasal region of the frame. Next, 510 the coordinates from the RGB facial recognition are retrieved and 512 fit to the resolution of the thermal camera. Then, the FIFO pipe is initiated to transfer the data 514 and to receive the coordinate data 516. The temperature is measured 518 from the coordinate data and an average temperature is calculated 520 from the five nasal coordinates and written as a text file. The text file is then read through a data analysis Python file 522 to find the number of breaths during the duration of the recording 524. The mobile application then receives and displays the number of breaths as a respiration rate 526 which is then logged and stored in the history 528.

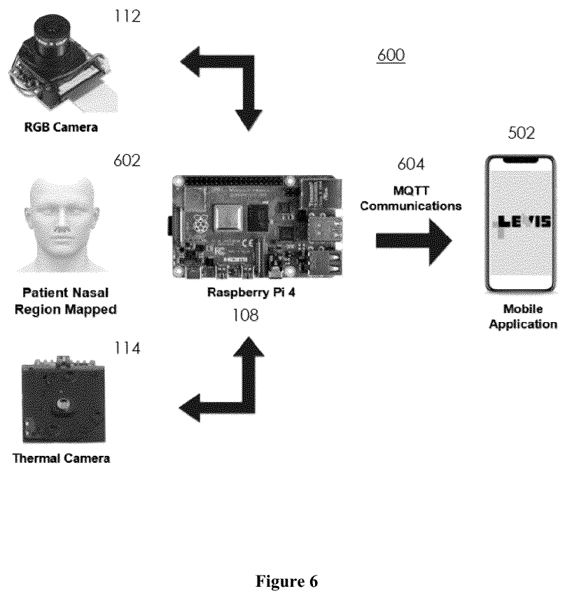

FIG. 6 represents a general schematic 600 of the communications between some of the components of the present invention. The input is the patient's nasal region 602 mapped from the RGB camera 112 and thermal camera 114. This data is sent to the Raspberry Pi board 108 and then using MQTT communications 604 the respiratory rate is then output to display on the cellular application 502.

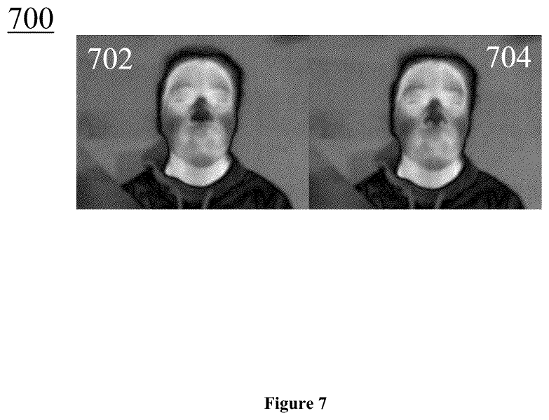

FIG. 7 is an example image from the thermal camera 114. The figure on the left 702 shows the user inhaling and the right FIG. 704 shows the user exhaling. The image displays increasing temperatures as warm colors and decreasing temperatures as cooler colors. Warm colors are seen around the nostril region of the image in 704 indicating the user is exhaling.

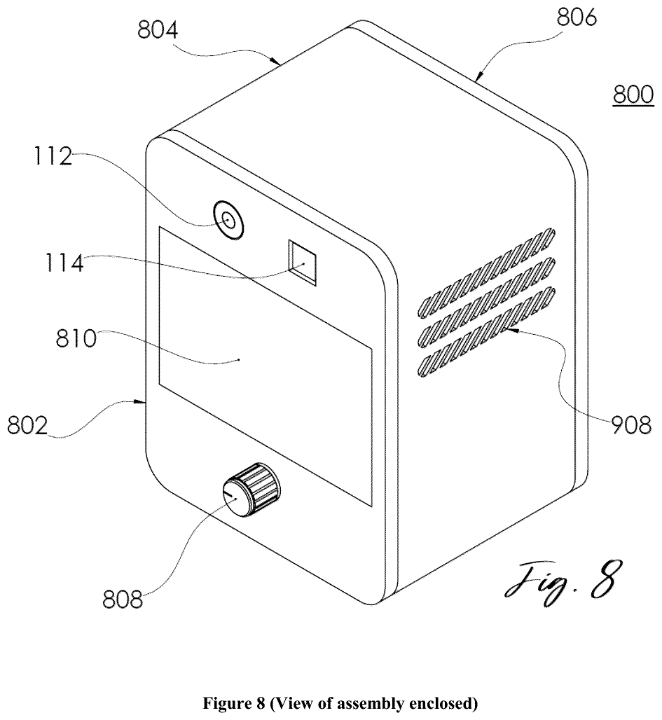

FIG. 8 illustrates another embodiment of the present invention in which the system is contained in a mobile unit 800. The mobile unit 800 is comprised of three different elements, a front, a middle and a back, 802, 804, and 806, respectively. Shown on the mobile unit 800 is an LCD display screen 810. There is also a hole for the RGB camera 112 and a hole for the thermal camera 114. A rotary encoder, 808, is used to control and navigate the device.

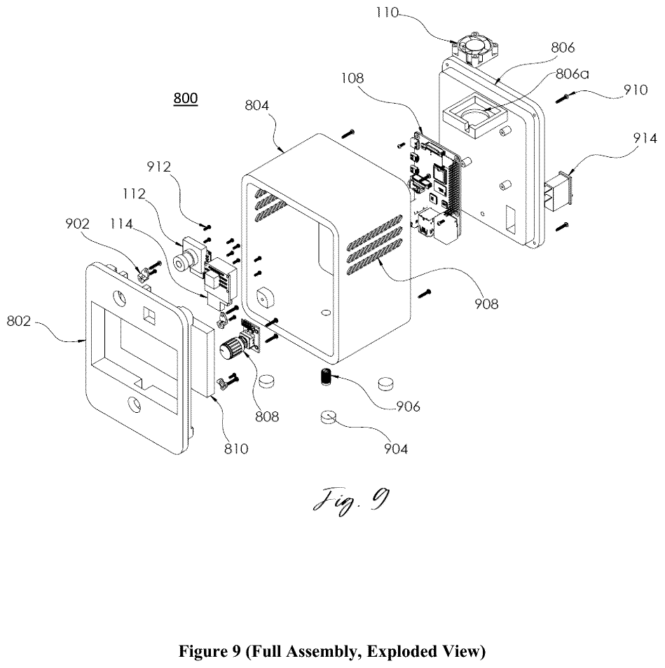

FIG. 9 is an exploded view of the mobile-unit 800 shown in FIG. 8. The exploded view 900 illustrates the major components shown in FIG. 1 and FIG. 8, as well as the mount pieces 902, the various assembly screws 912, the switch 914, and fan 110. Thread inserts 906 and threat feet 904 are also shown. A back fan mount 806a is also shown to mount fan 110.

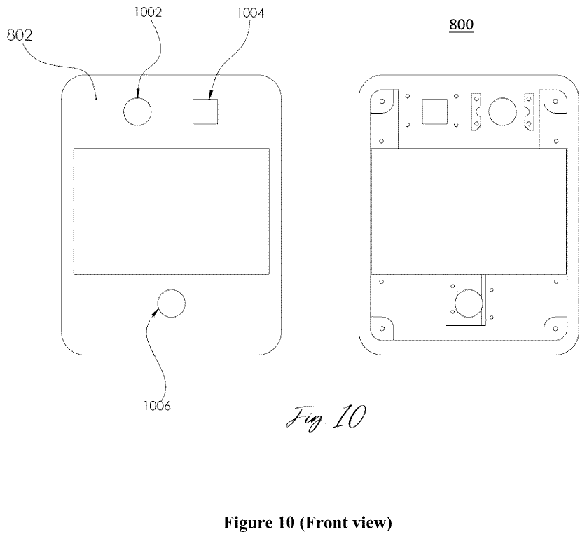

FIG. 10 shows the front 802 of the mobile unit 800. The housing features a hole 1002 for the RGB camera 112 (not shown) and a hole 1004 for the thermal camera 114 not shown, and a hole 1006 for the rotary encoder 808 (not shown).

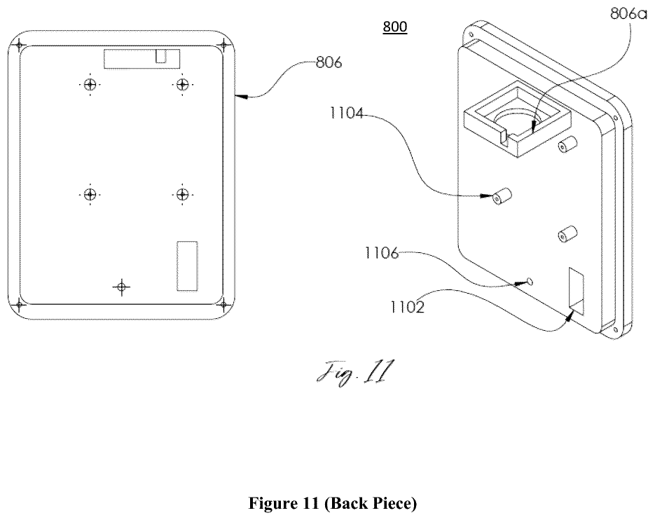

FIG. 11 is a drawing of the back 806 of a housing case for the portable, mobile unit 800. Shown is a fan mount 806a that is attached to the back 806 for the cooling of the Raspberry Pi board 108. Shown in the figure is the hole for switch 1102. The switch 914 is also shown and used for turning the device on and off.

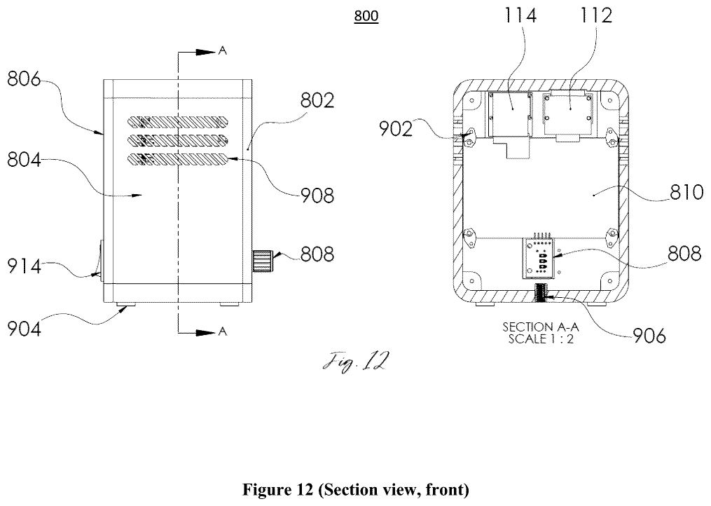

FIG. 12 is a drawing of the side (left) and section view of the front piece 2 (right) of the medical monitoring system with the portable, mobile unit 800. Shown in the side view is the switch 914, rotary encoder 808, rubber feet 904, thread insert, 906, and vent holes 908. Shown in the section view are the four LCD mount pieces 902, and rotary encoder 808.

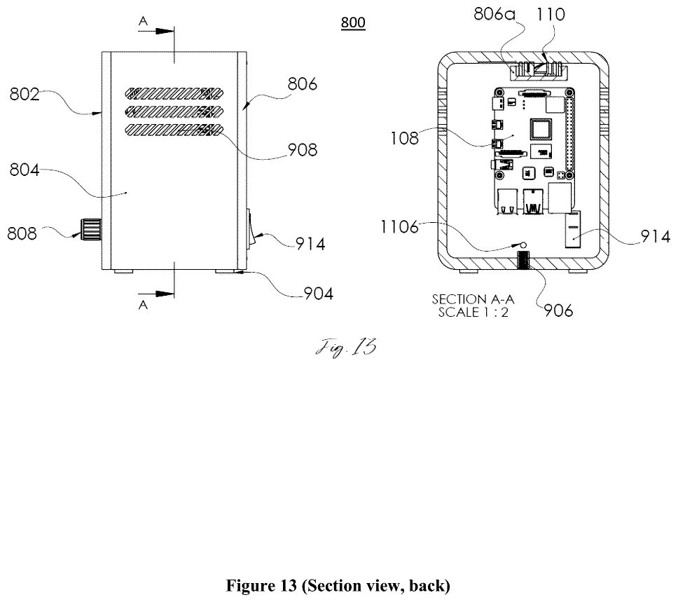

FIG. 13 is a drawing of the side (left) and section view of the back piece 6 (right) of the medical monitoring system with the portable, mobile unit 800. Shown in the side view is the switch 914, rotary encoder 808, rubber feet 904, threaded insert 906, and vent holes 908. Shown in the section view is the fan mount 806a and Raspberry Pi 108 as well as other components described previously.

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “medical monitoring device”, “medical monitoring system”, “device”, “mobile unit”, and “system” are interchangeable and refer to the present invention Respire 1.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

We claim:

1. A medical monitoring device comprising:
at least one visible imaging camera configured to obtain a visible image;
at least one thermal imaging camera configured to obtain a thermal image;
a processing unit configured to receive the visible image and the thermal image;
the processing unit further configured to locate and track a mouth and nasal region in the visible image and locate and track the mouth and nasal region in the thermal image, monitor and measure changes in temperature of the mouth and nasal region in the thermal image, and calculate a respiration rate and a body temperature; and,
an input/output device whereby a user controls the medical monitoring device, and the processing unit displays the respiratory rate and the body temperature.

2. The medical monitoring device of claim 1 wherein the processing unit may be any processing unit from the list consisting of microprocessor, raspberry pi, digital signal processor, application specific integrated circuit, and field programmable gate array.

3. The medical monitoring device of claim 1 wherein the processing unit is configured with software comprising machine learning to identify, locate, and track facial features in the visible image.

4. The medical monitoring device of claim 1 wherein the processing unit is configured with firmware comprising machine learning to identify, locate, and track facial features in the visible image.

5. The medical monitoring device of claim 1 wherein the visible imaging camera and the thermal imaging camera are contained in a camera housing configured to hold the visible imaging camera a fixed distance from the thermal imaging camera.

6. The medical monitoring device of claim 1 wherein the input/output device is selected from the group consisting of a touchscreen, a display screen with a rotary encoder, and an application installed on a smart phone remotely connected to the device.

7. The medical monitoring device of claim 1 wherein the medical monitoring device is contained in a portable, mobile unit.

8. A medical monitoring system comprising:
at least one visible imaging camera configured to obtain a visible image;
at least one thermal imaging camera configured to obtain a thermal image;
a processing unit configured to receive the visible image and the thermal image;
the processing unit further configured to locate and tracks a mouth and nasal region in the visible image and locate and track the mouth and nasal region in the thermal image, monitors and measures changes in temperature of the mouth and nasal region in the thermal image, wherefrom the processing unit calculates a respiration rate and a body temperature;
an input/output device whereby a user controls the function of the medical monitoring system, and the processing unit displays the respiratory rate and the body temperature;
an articulating arm configured to receive the input/output device, the visible imaging camera, and the thermal imaging camera; and,
the articulating arm further configured to receive a pole wherein the pole is attached to a base having a plurality of casters.

9. The medical monitoring system of claim 8 wherein the visible imaging camera and the thermal imaging camera are contained in a camera housing configured to hold the visible imaging camera and the thermal imaging camera.

10. The medical monitoring system of claim 8 wherein the input/output device is able to control the functions of the medical monitoring system remotely.

11. The medical monitoring system of claim 8 wherein the processing unit may be any processing unit from the list consisting of microprocessor, raspberry pi, digital signal processor, application specific integrated circuit, and field programmable gate array.

12. The medical monitoring system of claim 8 wherein the input/output device is selected from the group consisting of a touchscreen, a display screen with a rotary encoder, and an application installed on a smart phone remotely connected to the device.

13. The medical monitoring system of claim 8 further comprising a receiving unit from the list consisting of Bluetooth, MQTT, and Wi-Fi.

14. The medical monitoring system of claim 8 wherein the input/output device is an application installed on a smart phone remotely connected to the medical monitoring system.

15. A non-invasive method of monitoring vital signs comprising:
inputting data;
obtaining a visible video image;
locating a forehead, nasal, and mouth region in the visible video image;
obtaining a thermal video image;
correlating the forehead, nasal, and mouth region of the visible video with the same regions in the thermal video image;
measuring the temperature in the forehead, mouth, and nasal regions of the thermal video image and calculating respiration rate and body temperature; and,
displaying results of the respiration rate and body temperature on an output device.

16. The non-invasive method of monitoring vital signs of claim 15 wherein machine learning is used to locate the forehead, nasal, and mouth region in the visible video image.

17. The non-invasive method of monitoring vital signs of claim 15 wherein the respiration rate is calculated by dividing the number of temperature changes in the mouth and nasal region of the thermal video image by the duration of the thermal video image.
